(19) United States
(12) Patent Application Publication
(10) Pub. No.: US 2024/0408388 A1
(43) Pub. Date: Dec. 12, 2024

(54) FINGER MOVEMENT ASSISTING APPARATUS, FINGER MOVEMENT ASSISTING METHOD, AND PROGRAM

(71) Applicant: NIPPON TELEGRAPH AND TELEPHONE CORPORATION, Tokyo (JP)

(72) Inventor: Arinobu NIIJIMA, Musashino-shi, Tokyo (JP)

(73) Assignee: NIPPON TELEGRAPH AND TELEPHONE CORPORATION, Tokyo (JP)

(21) Appl. No.: 18/698,434

(22) PCT Filed: Oct. 6, 2021
(86) PCT No.: PCT/JP2021/037027

§ 371 Date: Apr. 4, 2024

(51) Int. Cl. A61N 1/36; A61N 1/372

(52) U.S. Cl. CPC A61N 1/36003; A61N 1/37247

(57) ABSTRACT

A finger exercise assistance device according to one aspect of the present invention includes an EMS (electrical muscle stimulation) control unit configured to control: a first EMS presentation unit configured to present a first EMS to a user's extensor muscle to contract the extensor muscle; and a second EMS presentation unit configured to present a second EMS to the user's flexor muscle that is an antagonist to the extensor muscle to contract the flexor muscle, wherein the EMS control unit is configured to drive the second EMS presentation unit to present the second EMS to the user's flexor muscle while the first EMS is presented to the user's extensor muscle, and the second EMS has a stronger intensity than the first EMS.

### TECHNICAL FIELD

The present invention relates to a technology for assisting a human with a finger exercise.

### BACKGROUND ART

There is a known method of guiding fingers to a specific posture by presenting electrical muscle stimulation (EMS) to muscles that move the fingers and causing the muscles to contract involuntarily, thereby assisting with learning of an exercise of human fingers. For example, NPL 1 discloses a method of extending and flexing a finger by presenting EMS to the extensor digitorum and flexor digitorum superficialis of the user's forearm. NPL 2 discloses a method of independently flexing each finger by presenting EMS to the lumbricals on the back of the hand.

### CITATION LIST

### Non Patent Literature

[NPL 1] Emi Tamaki, Takashi Miyaki and Jun Rekimoto, “PossessedHand: Techniques for Controlling Human Hands using Electrical Muscles Stimuli”, Proceedings of the SIGCHI Conference on Human Factors in Computing Systems, 2011.
[NPL 2] Akifumi Takahashi et al., “Increasing Electrical Muscle Stimulation's Dexterity by means of Back of the Hand Actuation”, Proceedings of the 2021 CHI Conference on Human Factors in Computing Systems, 2021.

### SUMMARY OF INVENTION

### Technical Problem

In a case where the EMS is presented to the forearm as in the method disclosed in NPL 1, it is difficult to independently control the second to fifth fingers. Because muscles moving fingers are dense in the forearm, even if stimulation is presented only to a specific muscle, the stimulation is also transmitted to adjacent muscles, and thus unintended fingers are moved simultaneously.

In a case where the EMS is presented to the back of the hand as in the method disclosed in NPL 2, each finger can be flexed independently, but the fingers cannot be extended. This is because there are no muscles on the back of the hand to extend the fingers. Therefore, even if EMS is presented on the back of the hand while the hand is clenched, the posture of the fingers cannot be changed.

An object of the present invention is to provide a technology that enables extension of the second to fifth fingers and flexion of any one of the second to fifth fingers independently.

### Solution to Problem

A finger exercise assistance device according to one aspect of the present invention includes an EMS (electrical muscle stimulation) control unit configured to control: a first EMS presentation unit configured to present a first EMS to a user's extensor muscle to contract the extensor muscle; and a second EMS presentation unit configured to present a second EMS to the user's flexor muscle that is an antagonist to the extensor muscle to contract the flexor muscle,
wherein the EMS control unit is configured to drive the second EMS presentation unit to present the second EMS to the user's flexor muscle while the first EMS is presented to the user's extensor muscle, and
the second EMS has a stronger intensity than the first EMS.

### Advantageous Effects of Invention

According to the present invention, it is possible to provide a technology that enables extension of the second to fifth fingers and flexion of any one of the second to fifth fingers independently.

### BRIEF DESCRIPTION OF DRAWINGS

FIG. 1 is a function block diagram illustrating a finger exercise assistance device according to the present embodiment.

FIG. 2 is a block diagram illustrating a hardware configuration of the finger exercise assistance device according to the present embodiment.

FIG. 3 is a diagram illustrating a user wearing an electrode pair shown in FIG. 2.

FIG. 4 is a flowchart illustrating a finger exercise assistance method according to an embodiment.

FIG. 5 is a diagram illustrating a posture in which only the third to fifth fingers are flexed according to the embodiment.

FIG. 6 is a diagram illustrating a posture in which all fingers are flexed as a comparative example.

### DESCRIPTION OF EMBODIMENTS

An embodiment of the present invention will be described below with reference to the drawings.

The embodiment relates to a technology for assisting a user with a finger exercise. In particular, the embodiment relates to a technology for guiding a user's fingers to a posture in which at least one of the user's second to fifth fingers is flexed and the remaining fingers are extended. This embodiment adopts electrical muscle stimulation (EMS) which is presented to muscles to cause them to contract involuntarily.

The technology according to the embodiment relates to presentation of the EMS to the user's extensor digitorum and lumbricals. The extensor digitorum is the extensor muscle that extends the second to fifth fingers. When the extensor digitorum contracts upon receiving EMS, the second to fifth fingers are extended. The lumbricals are antagonistic muscles to the extensor digitorum, and are also flexor muscles that flex each of the second to fifth fingers. When one of the lumbricals contracts due to the EMS, the user's finger corresponding to the lumbrical is flexed. The extensor digitorum and lumbricals form pairs of agonist and antagonistic muscles. The technology according to the embodiment relates to presentation of the EMS to the lumbrical corresponding to a specific finger (a finger desired to be flexed) of the user's while presenting the EMS to the extensor digitorum of the user. The intensity of EMS presented to the lumbrical is stronger than that presented to the extensor digitorum. This gives priority to flexion. Accordingly, a posture can be achieved in which a specific finger is flexed and the remaining fingers are extended.

### [Configuration]

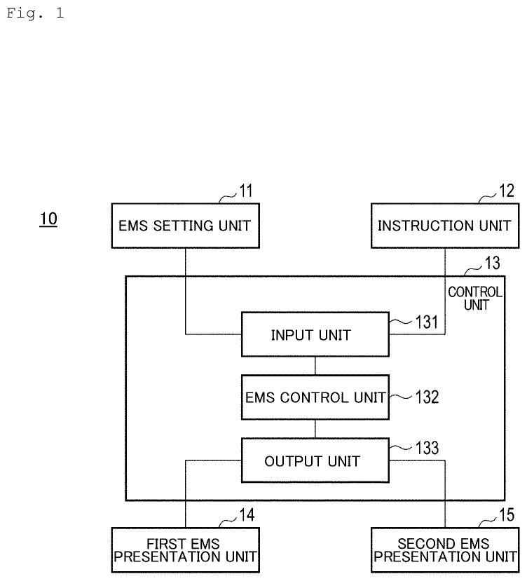

FIG. 1 schematically shows a finger exercise assistance device 10 according to one embodiment of the present invention. As shown in FIG. 1, the finger exercise assistance device 10 includes an EMS setting unit 11, an instruction unit 12, a control unit 13, a first EMS presentation unit 14 and a second EMS presentation unit 15.

The EMS setting unit 11 receives setting information including parameters related to EMS to be presented to the user from a human operator, and sends the setting information to the control unit 13. The setting information includes parameters related to EMS to be presented to the user's extensor digitorum and parameters related to EMS to be presented to the user's lumbrical. The parameters for EMS include frequency, current value, voltage value, and pulse width. The frequency, current value, voltage value, and pulse width are parameters for adjusting the stimulation intensity of the EMS. The parameters are set such that the intensity of EMS presented to the user's lumbrical is stronger than the intensity of EMS presented to the user's extensor digitorum. For example, the current value of EMS to be presented to the lumbrical is set to 12 mA, and the current value of EMS to be presented to the extensor digitorum is set to be 8 mA. The parameters for EMS may further include a time length. The time length is a parameter that indicates the duration for which EMS is presented.

The instruction unit 12 receives an instruction from the operator to designate a finger to be flexed, and sends an instruction signal indicating the finger designated by the operator to the control unit 13.

The control unit 13 controls the first EMS presentation unit 14 and the second EMS presentation unit 15. The control unit 13 includes an input unit 131, an EMS control unit 132 and an output unit 133.

The input unit 131 receives the setting information from the EMS setting unit 11 and sends the setting information to the EMS control unit 132. Furthermore, the input unit 131 receives the instruction signal from the instruction unit 12 and sends the instruction signal to the EMS control unit 132.

The EMS control unit 132 receives the setting information from the EMS setting unit 11 via the input unit 131. As described above, the setting information includes parameters related to EMS to be presented to the user's extensor digitorum and parameters related to EMS to be presented to the user's lumbrical. The EMS control unit 132 sets the parameters for EMS to be presented to the user's extensor digitorum in the first EMS presentation unit 14, and sets the parameters for EMS to be presented to the user's lumbrical in the second EMS presentation unit 15.

The EMS control unit 132 receives the instruction information from the instruction unit 12 via the input unit 131. The EMS control unit 132 drives the first EMS presentation unit 14 and the second EMS presentation unit 15 on the basis of the received instruction signal. For example, in a case where the instruction signal indicates that the user's second finger should be flexed, the EMS control unit 132 drives the first EMS presentation unit 14 while driving the second EMS presentation unit 15 to present the EMS to the lumbrical corresponding to the second finger. For example, in a case where the instruction signal indicates that the user's second and third fingers should be flexed, the EMS control unit 132 drives the first EMS presentation unit 14 while driving the second EMS presentation unit 15 to present the EMS to the lumbrical corresponding to the second finger and the lumbrical corresponding to the third finger. The EMS control unit 132 generates a first drive signal for driving the first EMS presentation unit 14 and a second drive signal for driving the second EMS presentation unit 15.

The output unit 133 outputs the first drive signal generated by the EMS control unit 132 to the first EMS presentation unit 14, and outputs the second drive signal generated by the EMS control unit 132 to the second EMS presentation unit 15.

The first EMS presentation unit 14 presents the EMS to the user's extensor digitorum under the control of the control unit 13. While receiving the first drive signal from the control unit 13, the first EMS presentation unit 14 generates the EMS according to the parameters set by the control unit 13, and presents the EMS to the user's extensor digitorum.

The second EMS presentation unit 15 presents the EMS to at least one of the user's lumbricals under the control of the control unit 13. While receiving the second drive signal from the control unit 13, the second EMS presentation unit 15 generates the EMS according to the parameters set by the control unit 13, and presents the EMS to the user's lumbrical.

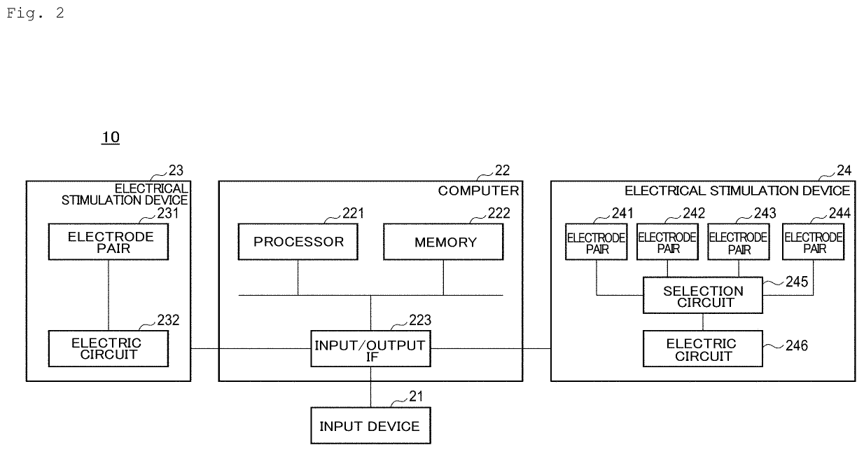

The EMS setting unit 11, the instruction unit 12, the control unit 13, the first EMS presentation unit 14 and the second EMS presentation unit 15 are herein collectively referred to as the finger exercise assistance device. As will be described later with reference to FIG. 2, the EMS setting unit 11, the instruction unit 12, the control unit 13, the first EMS presentation unit 14 and the second EMS presentation unit 15 can be implemented as a plurality of hardware devices separated from each other. The whole device may be referred to as a finger exercise assistance system, and the control unit 13 alone may be referred to as the finger exercise assistance device.

FIG. 2 schematically illustrates a hardware configuration example of the finger exercise assistance device 10. As shown in FIG. 2, the finger exercise assistance device 10 includes an input device 21, a computer 22, an electrical stimulation device 23 and an electrical stimulation device 24. The input device 21 implements the EMS setting unit 11 and the instruction unit 12 illustrated in FIG. 1, the computer 22 implements the control unit 13 illustrated in FIG. 1, the electrical stimulation device 23 implements the first EMS presentation unit 14 illustrated in FIG. 1, and the electrical stimulation device 24 implements the second EMS presentation unit 15 illustrated in FIG. 1.

The input device 21 includes equipment operated by the operator and receives inputs from the operator. The input device 21 is used to input the setting information and the instruction designating a finger to be flexed. For example, the input device 21 includes a keyboard, mouse, and buttons.

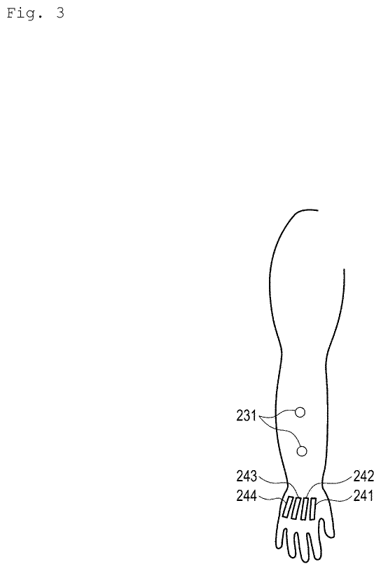

The electrical stimulation device 23 is configured to apply electrical stimulation to a muscle corresponding to a specific part of the user. As one example, the electrical stimulation device 23 includes an electrode pair 231 and an electric circuit 232 connected to the electrode pair 231. The two electrodes of the electrode pair 231 may be provided on individual electrode pads. The electrode pair 231 may be provided on a single electrode pad so that the two electrodes are kept a predetermined distance apart from each other. The electric circuit 232 generates an electrical stimulation signal (e.g. pulsed current signal) and applies the electrical stimulation signal to the user via the electrode pair 231. As shown in FIG. 3, the electrode pair 231 is attached to the user's forearm. In particular, the electrode pair 231 is attached to the area of the user's forearm opposite to the extensor digitorum to apply the electrical stimulation to the user's extensor digitorum.

Referring to FIG. 2 again, the electrical stimulation device 24 is configured to apply electrical stimulation to a muscle corresponding to the user's specific site. As one example, the electrical stimulation device 24 includes four electrode pairs 241, 242, 243 and 244, a selection circuit 245, and an electric circuit 246. The electrode pairs 241 to 244 are connected to the electric circuit 246 via the selection circuit 245. The two electrodes of each electrode pair 241, 242, 243 or 244 may be provided on individual electrode pads. Each of the electrode pair 241, 242, 243 and 244 may be provided on a single electrode pad so that the two electrodes are kept a predetermined distance apart from each other. The selection circuit 245 selects one or more of electrode pairs 241 to 244 and connects the one or more selected electrode pairs to the electric circuit 246. The electric circuit 246 generates an electrical stimulation signal (e.g. pulsed current signal) and applies the electrical stimulation signal to the user via the one or more selected electrode pairs. As shown in FIG. 3, the electrode pairs 241 to 244 are attached to the user's forearm. In particular, the electrode pair 241 is attached to the back of the user's hand in a region opposite to the lumbrical of the second finger to apply the electrical stimulation to the lumbrical of the user's second finger; the electrode pair 242 is attached to the back of the user's hand in a region opposite to the lumbrical of the third finger to apply the electrical stimulation to the lumbrical of the user's third finger; the electrode pair 243 is attached to the back of the user's hand in a region opposite to the lumbrical of the fourth finger to apply the electrical stimulation to the lumbrical of the user's fourth finger; and the electrode pair 244 is attached to the back of the user's hand in a region opposite to the lumbrical of the fifth finger to apply the electrical stimulation to the lumbrical of the user's fifth finger.

In the example shown in FIG. 2, a single electrical stimulation device is used to selectively stimulate four lumbricals. In another example, multiple (e.g. four) electrical stimulation devices may be used to selectively stimulate four lumbricals.

The computer 22 may be, but is not limited to, a microcontroller. A computer 22 includes a processor 221, a memory 222, and an input/output interface 223. The processor 221 is connected to the memory 222 and the input/output interface 223 and exchanges signals with the memory 222 and the input/output interface 223.

A processor 221 is one example of a processing circuit. The processor 221 is, for example, an integrated circuit such as a central processing unit (CPU).

The memory 222 stores various types of data and programs to be executed by the processor 221 such as a finger exercise assistance program. Each program includes a plurality of computer-executable instructions. The processor 221 executes the program stored in the memory 222. The finger exercise assistance program, when executed by the processor 221, causes the processor 221 to perform a series of processes described with respect to the control unit 13. In other words, the processor 221 functions as the input unit 131, the EMS control unit 132 and the output unit 133 according to the finger exercise assistance program.

The program may be provided to the computer 22 under a state in which the program is stored in a computer-readable recording medium. In this case, the computer 22 includes a drive that reads data from the recording medium, and acquires the program from the recording medium. Examples of the recording medium include a magnetic disk, an optical disk (such as CD-ROM, CD-R, DVD-ROM, or DVD-R), a magneto-optical disk (such as MO), and a semiconductor memory. Further, the program can also be provided through a network. Specifically, the program may be stored in a server on a network, and the computer 22 may download the program from the server.

The input/output interface 223 is an interface for connecting with the input device 21, the electrical stimulation device 23 and the electrical stimulation device 24. The processor 221 receives the setting information and the instruction signals from the input device 21 via the input/output interface 223. The processor 221 transmits the first drive signal to the electrical stimulation device 23 via the input/output interface 223. The processor 221 transmits the second drive signal to the electrical stimulation device 24 via the input/output interface 223.

### [Operations]

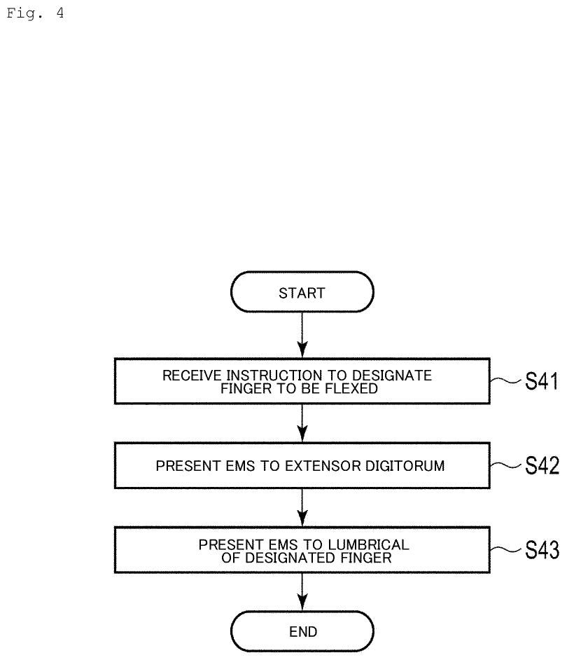

FIG. 4 schematically shows a finger exercise assistance method executed by the finger exercise assistance device 10.

In a step S41 of FIG. 4, the finger exercise assistance device 10 receives the instruction to designate a finger to be flexed from the operator. For example, the operator inputs the instruction to designate a finger to be flexed to the instruction unit 12, and the input unit 131 acquires the instruction input by the operator.

In step S42, the finger exercise assistance device 10 presents EMS having a first stimulation intensity to the user's extensor digitorum to extend the user's second to fifth fingers. The EMS control unit 132 drives the first EMS presentation unit 14 to present the EMS having the first stimulation intensity to the user's extensor digitorum.

In step S43, the finger exercise assistance device 10 presents EMS having a second stimulation intensity stronger than the first stimulation intensity to the user's lumbrical corresponding to the designated finger to flex the designated finger. The EMS control unit 132 drives the second EMS presentation unit 15 to present the EMS having the second stimulation intensity to the designated finger's lumbrical. The EMS control unit 132 drives the first EMS presentation unit 14 while driving the second EMS presentation unit 15. That is, the finger exercise assistance device 10 presents the EMS to the lumbrical of the designated finger while presenting the EMS to the user's extensor digitorum. The intensity of EMS presented to the lumbrical is stronger than that presented to the extensor digitorum. Consequently, flexion is given priority in simultaneous stimulation of extension and flexion. Accordingly, the posture can be maintained in which only the designated finger is flexed and the remaining fingers are extended.

### [Effects]

In the finger exercise assistance device 10, the first EMS presentation unit 14 is configured to present EMS to the user's extensor digitorum to contract the extensor digitorum, the second EMS presentation unit 15 is configured to independently present EMS to the plurality of lumbricals to contract at least one of lumbricals of the user, and the EMS control unit 132 is configured to controls the first EMS presentation unit 14 and the second EMS presentation unit 15. The EMS control unit 132 drives the second EMS presentation unit 15 to present the EMS to the lumbrical corresponding to a specific finger while the EMS is presented to the user's extensor digitorum. The EMS presented to the lumbrical has stronger intensity than the EMS presented to the extensor digitorum. Thus, the present invention enables extension of the second to fifth fingers and flexion of any one of the second to fifth fingers independently. For example, it is possible to change a state where the hand is clenched (the second to fifth fingers are flexed) to a state where a specific finger (specifically, a finger designated by the operator) is flexed and the remaining fingers are extended. Further, the user can grip an object while putting strength into a specific finger only.

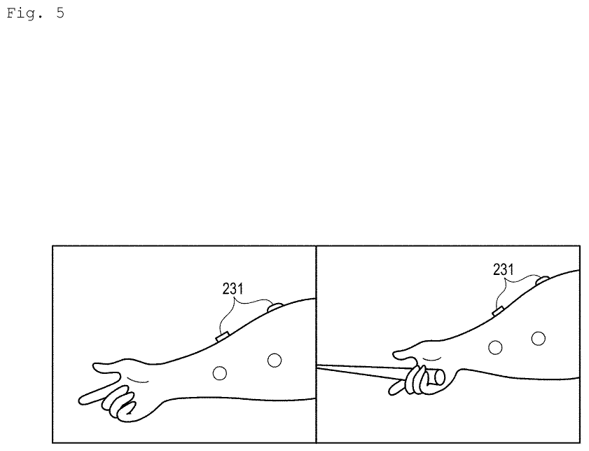

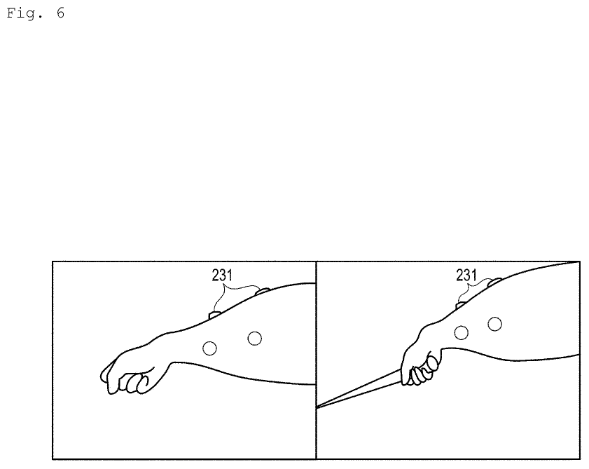

Referring to FIGS. 5 and 6, a description will be given of a case of verifying the change in finger posture when EMS is actually presented to a plurality of muscles. Electrode pairs for presenting EMS were respectively attached to the set of lumbricals corresponding to the third to fifth fingers, the extensor digitorum, and the flexor pollicis longus for flexion of the thumb. In a case where EMS (current: 12 mA, frequency: 200 Hz, pulse width: 200 μs) is presented to the lumbrical after presenting EMS (current: 8 mA, frequency: 200 Hz, pulse width: 200 μs) to the extensor digitorum, only the third to fifth fingers are flexed as shown in FIG. 5. On the other hand, in a case where EMS (current: 12 mA, frequency: 200 Hz, pulse width: 200 μs) is presented to the lumbrical and flexor pollicis longus, all fingers are clenched as shown in FIG. 6.

According to the embodiment of the present invention, it is possible to generate fine differences in finger postures, such as which fingers are used to grip an object. The finger exercise assistance device 10 can be used for finger exercise learning such as rehabilitation.

### [Modifications]

In the embodiment stated above, the user's second to fifth fingers are selectively flexed based on the instruction from the operator. In other words, in the embodiment described above, the second to fifth fingers are subjected to flexion control. In another embodiment, one, two or three fingers out of the second to fifth fingers may be subjected to flexion control.

For example, in a case where only the second finger is subjected to flexion control, it is sufficient that the second EMS presentation unit 15 is configured to present EMS to the lumbrical corresponding to the user's second finger. In this case, the electrical stimulation device 24 used as the second EMS presentation unit 15 may be provided with one electrode pair, which is attached to the skin surface opposite to the lumbrical of the second finger. It is not necessary for the operator to designate the finger to be flexed, and the instruction unit 12 receives the instruction to start assistance for finger exercise from the operator.

For example, in a case where only the second finger and the third finger are to be subjected to flexion control, the second EMS presentation unit 15 may be configured to individually present EMS to the lumbricals each corresponding to the second finger and the third finger of the user.

Further, for example, in a case where a set of the second finger and the third finger is to be subjected to flexion control (the second and third fingers do not need to be individually controlled), the second EMS presentation unit 15 may be configured to present EMS to the lumbricals each corresponding to the second and third fingers of the user. In this case, one electrode pair may be used to present EMS to two lumbricals each corresponding to the second and the third finger.

Note that the present invention is not limited to the embodiments described above and can variously be modified at an execution stage within a scope not departing from the gist of the present invention. In addition, the embodiments may be combined as appropriate, and in such a case, combined effects can be achieved. Furthermore, the above-described embodiment includes various inventions and various inventions can be extracted by a combination selected from a plurality of disclosed components. For example, if the problem can be solved and the effect can be obtained even if some components are deleted from all the components shown in the embodiment, the configuration in which these components are deleted can be extracted as an invention.

### REFERENCE SIGNS LIST

10: Finger exercise assistance device
11: EMS setting unit
12: Instruction unit
13: Control unit
131: Input unit
132: EMS control unit
133: Output unit
14: First EMS presentation unit
15: Second EMS presentation unit
21: Input device
22: Computer
221: Processor
222: Memory
223: Input/output interface
23: Electrical stimulation device
231: Electrode pair
232: Electric circuit
24: Electrical stimulation device
241 to 244: Electrode pair
245: Selection circuit
246: Electric circuit

1. A finger exercise assistance device, comprising:
EMS (electrical muscle stimulation) control circuitry configured to control:
first EMS presentation circuitry configured to present a first EMS to a user's extensor muscle to contract the extensor muscle; and
second EMS presentation circuitry configured to present a second EMS to the user's flexor muscle that is an antagonist to the extensor muscle to contract the flexor muscle,
wherein the EMS control circuitry is configured to drive the second EMS presentation circuitry to present the second EMS to the user's flexor muscle while the first EMS is presented to the user's extensor muscle, and
the second EMS has a stronger intensity than the first EMS.

2. The finger exercise assistance device according to claim 1, wherein:
the extensor muscle includes extensor digitorum, and the flexor muscle includes lumbrical.

3. The finger exercise assistance device according to claim 2, further comprising:
an input interface to acquire an instruction designating a finger to be flexed among a plurality of fingers,
wherein:
the flexor muscle includes a plurality of lumbricals corresponding to each of the plurality of fingers, and
the EMS control circuitry is configured to drive the second EMS presentation circuitry to present the second EMS to a lumbrical corresponding to the designated finger among the plurality of lumbricals.

4. The finger exercise assistance device according to claim 3, wherein the EMS control circuitry is configured to:
drive the first EMS presentation circuitry to present the first EMS to the extensor digitorum in response to the acquired instruction; and
drive the second EMS presentation circuitry to present the second EMS to the lumbrical corresponding to the designated finger while the first EMS is presented to the extensor digitorum.

5. A finger exercise assistance method, comprising:
presenting a first EMS (electrical muscle stimulation) to a user's extensor muscle to contract the extensor muscle; and
presenting a second EMS to the user's flexor muscle that is an antagonist to the extensor muscle to contract the flexor muscle,
wherein:
the presenting the second EMS to the user's flexor muscle is performed while the presenting the first EMS to the user's extensor muscle, and
the second EMS has a stronger intensity than the first EMS.

6. A non-transitory computer readable medium storing a program for causing a computer to perform the method of claim 5.
